(19) United States
(12) Patent Application Publication
(10) Pub. No.: US 2010/0137927 A1
(43) Pub. Date: Jun. 3, 2010

(54) MULTIFUNCTIONAL CARDIAC PACEMAKER SYSTEM

(76) Inventor: TENGIZ TKEBUCHAVA, NEWTON, MA (US)

Correspondence Address: AKC PATENTS, 215 GROVE ST., NEWTON, MA, 02466, US

(21) Appl. No.: 12/628,327

(22) Filed: Dec. 1, 2009

Related U.S. Application Data
(60) Provisional application No. 61/118,887, filed on Dec. 1, 2008.

(51) Int. Cl. A61N 1/362

(52) U.S. Cl. 607/3; 607/37; 607/32

(57) ABSTRACT

A pacemaker system 100 includes a pacemaker device 160, cardiac leads 120 and 150, guide catheter 110, an ultrasound transmitter 133 and an ultrasound receiver 130. Cardiac lead 150 is implanted in the right atrium (RA) 82 and includes an electrode 152 at its distal end that is actively fixed into location 102 of the right atrium 82. Electrode 152 is used for pacing of the RA. Cardiac lead 120 is implanted in the right ventricle (RV) 84 and includes two separate electrodes. A first electrode 140 is actively fixed into location 101 close to the apex 98 of the right ventricle 84 and is used for pacing, sensing and/or defibrillating of the RV. A second electrode 130 perforates the apex 98 of the right ventricle 84 and is actively fixed into the apex 99 of the left ventricle (LV) 86. Electrode 130 is used for pacing, sensing and/or defibrillating of the LV.

### CROSS REFERENCE TO RELATED CO-PENDING APPLICATIONS

This application claims the benefit of U.S. provisional application Ser. No. 61/118,887 filed on Dec. 1, 2009 and entitled CARDIAC PACEMAKER SYSTEM FOR BIVENTRICULAR PACING, which is commonly assigned, and the contents of which are expressly incorporated herein by reference.

### FIELD OF THE INVENTION

The present invention relates to a multifunctional cardiac pacemaker system and a method of treating cardiac pathologies with this multifunctional cardiac pacemaker system.

### BACKGROUND OF THE INVENTION

The human heart is a muscular organ that pumps blood throughout the blood vessels.

The heart is located between the lungs in the middle of the chest, behind and slightly to the left of the sternum. A double-layered membrane called the pericardium surrounds the heart like a sac. The outer layer of the pericardium surrounds the roots of the heart's major blood vessels and is attached by ligaments to the spinal column, diaphragm, and other parts of the body. The inner layer of the pericardium (also called epicardium) is attached to the heart muscle (also call myocardium). A coating of fluid separates the two layers of membrane, letting the heart move as it beats, yet still be attached to the body.

Referring to FIG. 1, the human heart 80 includes four chambers 82, 84, 86, 88. The upper chambers 82, 88 are the right and left atria, and the lower chambers 84, 86 are the right and left ventricles. The left and right atria 88, 82 and the left and right ventricles 86, 84 are separated by the septum 105, i.e., a wall of muscle. The function of the right side of the heart is to collect de-oxygenated blood from the body into the right atrium 82 and then pump it via the right ventricle 84 into the lungs where it becomes oxygenated. The function of the left side of the heart is to collect the oxygenated blood from the lungs into the left atrium 88, then move it into the left ventricle 86 and from there pump it out to the body. The left ventricle 86 is the largest and strongest chamber in the heart. The left ventricle's chamber walls are about a half-inch thick and as they contract they apply enough force to push blood through the aortic valve 87 into the aorta 91 and then into the body. The right atrium 82 receives blood from the body through the superior vena cava 81 and inferior vena cava 94 and pumps it into the right ventricle 84 via the pulmonary valve 83. The right ventricle 84 pumps the blood into the pulmonary artery 92 via the tricuspid valve 85. The left atrium 88 receives blood from the lungs via the pulmonary vein 93 and pumps it into the left ventricle via the mitral valve 89. The left ventricle pumps the blood into the aorta 91 via the aortic valve 87. The heart muscle is supplied with blood via the coronary arteries (not shown) and is drained via the coronary veins. The coronary sinus (not shown) receives most of the blood from the heart and empties it into the right atrium.

Electrical signals (impulses) from the heart muscle (the myocardium) cause the heart to contract (heart beat). These electrical signals begin in the sinoatrial (SA) node, 96 located at the top of the right atrium 82, shown in FIG. 2. When an electrical signal is released from the SA node 96 (also called heart's natural pacemaker), it causes the right and left atria 82, 88 to contract. The contraction of the right atrium 82 pushes blood through the tricuspid valve 83 into the right ventricle 84. The contraction of the left atrium 88 pushes blood through the mitral valve 89 into the left ventricle 86. The electrical signal then passes through the atrioventricular (AV) node 97. The AV node 97 checks the signal and sends it through the heart muscle fibers to the apexes 98, 99 of the right and left ventricles 84, 86, respectively, causing them to contract (systole). During the systole, the tricuspid valve 83 and mitral valve 89 shut tight to prevent a backflow of blood, the pulmonary valve 85 and aortic valve 87 are pushed open and blood is pushed from the right ventricle 84 into the lungs to pick up oxygen, and oxygen-rich blood flows from the left ventricle 86 to the heart and other parts of the body. After blood moves into the pulmonary artery 92 and the aorta 91, the right and left ventricles 84, 86 relax, and the pulmonary 85 and aortic 87 valves close. The lower pressure in the ventricles 84, 86 causes the tricuspid 83 and mitral 89 valves to open, and the cycle begins again. This series of contractions is repeated over and over again, increasing during times of exertion and decreasing when the person is at rest. The heart normally beats about 60 to 80 times a minute when the person is at rest. Abnormal heartbeats (arrhythmias) are indications of heart pathologies.

Heart pathologies that involve abnormal heartbeats include atrial or ventricular tachycardia, brachycardia, fibrilation, flutter, premature contractions, and pathologies of the hearts conduction system (bradyarrhythmias). Several of these pathologies may be treated with an artificial pacemaker. An artificial pacemaker (pacemaker) is a medical device that sends small electrical impulses to the heart muscle to maintain a normal heart rate.

Referring to FIG. 3, a pacemaker system 70 is implanted surgically under the skin, usually below the left clavicle. The pacemaker system 70 includes a pulse generator 160, which houses a battery and a computer (not shown), and cardiac leads 120, 150 170 (wires) that send impulses from the pulse generator 160 to the heart muscle, as well as sense the heart's electrical activity. The wires 120, 150, 170 are implanted through the subclavian veins 106. Pacemakers are mostly used to prevent the heart from beating too slowly. Newer pacemakers may have additional features that are designed to help with the management of arrhythmias, optimize heart-rate-related functions and improve synchronization. Some devices combine a pacemaker and a defibrillator in a single implantable device. Others have multiple electrodes stimulating differing positions within the heart to improve synchronization of the right and left ventricles of the heart (biventricular stimulation). A conventional cardiac resynchronization therapy (CRT) system includes three separate cardiac leads 120, 150, 170, as shown in FIG. 3. Cardiac lead 150 is placed into the right atrium and stimulates the right atrium 82, cardiac lead 120 is placed into the right ventricle and stimulates the right ventricle 84 and lead 170 is placed into the left ventricle and stimulates the left ventricle 86.

The implantation of multiple electrodes into the heart increases the duration of the surgery and the risk for infections. The left ventricle stimulation lead 170 is usually introduced through cannulation of the coronary sinus. However, cannulation of the coronary sinus may cause blood flow, obstructions and problems with the fixation of the lead. In some cases, introduction of lead 170 via cannulation of the coronary sinus is impossible and lead 170 is introduced via surgery. Furthermore, the implanted cardiac leads may become dislodged either during the implantation procedure or by normal physiological activity of the patient. A clip-on tool 180 and/or other fixation devices, such as screws or anchors are used for the fixation of the leads and the prevention of lead dislodgement. The installation of these additional components increase the duration and complexity of the surgery.

Accordingly, there is a need for an improved cardiac lead system that facilitates biventricular stimulation without the need of implanting multiple leads and without the need for additional fixation components.

### SUMMARY OF THE INVENTION

In general, in one aspect, the invention features a cardiac pacemaker system for biventricular pacing including a pacemaker device, and first and second cardiac leads. The pacemaker device comprises a pulse generator for producing cardiac stimulating pulses. The first cardiac lead is connected to the pulse generator and comprises first and second electrodes and is shaped and dimensioned to be implanted in the right cardiac ventricle. The first electrode comprises first fixation means for actively fixing the first electrode to the apex of the right cardiac ventricle. The second electrode comprises means for penetrating the apex of the right cardiac ventricle, means for entering into the left cardiac ventricle and second fixation means for actively fixing the second electrode to the apex of the left cardiac ventricle. The second cardiac lead is also connected to the pulse generator and comprises a third electrode and is shaped and dimensioned to be implanted in the right cardiac atrium. The stimulating pulses are transmitted to the right and left cardiac ventricles via the first cardiac lead and to the right cardiac atrium via the second cardiac lead and stimulate the apex of the right cardiac ventricle, the apex of the left cardiac ventricle and the first location of the right cardiac atrium via the first, second and third electrodes, respectively.

Implementations of this aspect of the invention may include one or more of the following features. The first cardiac lead comprises a flexible hollow tube comprising a proximal end and a distal end and the hollow tube defines a lumen extending between the proximal and distal ends and is dimensioned to house a conductive lead connecting the pulse generator to the first and second electrodes. The conductive lead may be a conductive heat shrinkable polymer. The second electrode comprises a cone-shaped body having a sharp tip end for penetrating the apex of the right cardiac ventricle and a cavity containing the second fixation means. The second fixation means comprise first and second foldable wings, a screw-driven mechanism for folding and unfolding the first and second foldable wings and a stylet used to activate the screw-driven mechanism and to push the second electrode into the left cardiac ventricle. The stylet is inserted through the lumen and is attached to the screw-driven mechanism. The stylet may be attached to the screw-driven mechanism via a clockwise rotation and activates the screw-driven mechanism via a counter-clockwise rotation. The stylet may be pushed forward to be attached to the screw-driven mechanism and may be pulled back to activate the screw-driven mechanism. The first cardiac lead further comprises an ultrasound transmitter at its distal end and an ultrasound receiver at its proximal end. The ultrasound transmitter is located and oriented so that it transmits ultrasound waves that pass through the cardiac left ventricle and left atrium and are modulated by the cardiac rhythm prior to being received by the ultrasound receiver. The modulated ultrasound waves comprise information about at least one of rhythm of left and right cardiac ventricles, heart rate, left ventricular ejection fraction, left ventricular ejection time, left ventricular pre-ejection time, global interval CO interval, EA interval, Q-A2 interval, aortic velocity time integrals LVdp/dt, CI, cardiac output and fractional shortening. The pacemaker device comprises a processor for analyzing the information and providing feedback control to the pulse generator. The pacemaker device further comprises a wireless transmitter for transmitting the information wirelessly to a remote location for monitoring purposes. The cardiac pacemaker system may further include a guide catheter. The guide catheter comprises a flexible hollow tubular body dimensioned to house the first and second cardiac leads and the stylet and to be implanted into a mammalian heart via the subclavian vein. The tubular body comprises a distal end that is bendable and forms an angle with the tube main axis and the angle is controlled via a control located at the proximal end of the tubular body. The guide catheter may further comprise radiographic position markers for 3-D visualization and positioning. The guide catheter may further comprise diagnostic devices for determining the condition of the surrounding tissue. The pacemaker device may further comprise a drug injection port that connects to the flexible hollow tube of the first cardiac lead and is used to inject drugs, stem cells dies, genes or other medication substance to the heart muscle of the right ventricle and/or the left ventricle. The first and second electrodes may include apertures for delivering the injected drugs to the right and left cardiac ventricles, respectively.

In general, in another aspect, the invention features a method of stimulating a mammalian heart via a single pacing/sensing cardiac pacemaker system. The method includes the following steps. First, providing a pacemaker system comprising a pacemaker device, first and second cardiac leads and a guide catheter. The pacemaker device comprises a pulse generator for producing cardiac stimulating pulses. The first cardiac lead is connected to the pulse generator and comprises first and second electrodes. The second cardiac lead is connected to the pulse generator and comprises a third electrode. The guide catheter comprises a flexible hollow tubular body dimensioned to house the first and second cardiac leads and a stylet. Next, inserting the guide catheter into a right cardiac ventricle via the subclavian vein. Next, inserting the first cardiac lead through the guide catheter into the right cardiac ventricle and actively fixing the first electrode to the apex of the right cardiac ventricle with first fixation means. Next, inserting the stylet into the first cardiac lead and attaching the stylet to the second electrode. Next, pushing the second electrode with the stylet through the apex of the right cardiac ventricle and position it at the apex of the left cardiac ventricle. Next, activating the second electrode's active fixation mechanism with the stylet and fixing the second electrode to the apex of the left cardiac ventricle with second fixation means. Next, inserting the second cardiac lead through the guide catheter into the right cardiac atrium. Finally, initiating pacing of the right cardiac ventricle, left cardiac ventricle and right cardiac atrium via the first second and third electrodes, respectively.

Implementations of this aspect of the invention may include one or more of the following features. The first cardiac lead comprises a flexible hollow tube comprising a proximal end and a distal end and the hollow tube defines a lumen extending between the proximal and distal ends and is dimensioned to house a conductive lead connecting the pulse generator to the first and second electrodes. The second electrode comprises a cone-shaped body having a sharp tip end for penetrating the apex of the right cardiac ventricle and a cavity containing the second fixation means. The second fixation means comprise first and second foldable wings, a screw-driven mechanism for folding and unfolding the first and second foldable wings and the stylet is inserted through the lumen and is attached to the screw-driven mechanism and is used to activate the screw-driven mechanism and to push the second electrode into the left cardiac ventricle. The first cardiac lead may further comprise an ultrasound transmitter at its distal end and an ultrasound receiver at its proximal end and the ultrasound transmitter is located and oriented so that it transmits ultrasound waves that pass through the cardiac left ventricle and left atrium and are modulated by the cardiac rhythm prior to being received by the ultrasound receiver. The modulated ultrasound waves comprise information about at least one of rhythm of left and right cardiac ventricles, heart rate, left ventricular ejection fraction, left ventricular ejection time, left ventricular pre-ejection time, global interval CO interval, EA interval, Q-A2 interval, aortic velocity time integrals LVdp/dt, CI, cardiac output and fractional shortening and the pacemaker device comprises a processor for analyzing the information and providing feedback control to the pulse generator. The pacemaker device may further comprise a wireless transmitter for transmitting the information wirelessly to a remote location for monitoring purposes. The tubular body of the guide catheter comprises a distal end that is bendable and forms an angle with the tube main axis and wherein the angle is controlled via a control located at the proximal end of the tubular body. The guide catheter further comprises radiographic position markers for 3-D visualization and positioning. The pacemaker device further comprises a drug injection port that connects to the flexible hollow tube of the first cardiac lead and is used to inject drugs, stem cells dies, genes or other medication substance to the heart muscle of the right ventricle and/or the left ventricle.

### BRIEF DESCRIPTION OF THE DRAWINGS

FIG. 1 is a cross-sectional view of a human heart;

FIG. 2 depicts the electrical conduction system of the human heart;

FIG. 3, depicts a prior art pacemaker system;

FIG. 4 depicts an embodiment of a cardiac pacemaker system of this invention;

FIG. 5 depicts the placement of the electrode of the pacemaker system of FIG. 4 at the apex of the left ventricle;

FIG. 6 depicts the ultrasound feedback signal of the pacemaker system of FIG. 4;

FIG. 7 depicts the active fixation of the LV electrode at the apex of the left ventricle;

FIG. 8 is a schematic diagram of the distal end of the cardiac lead of the pacemaker system of FIG. 4;

FIG. 9 depicts the unfolding of a set of wings for the fixation of the cardiac lead electrode in the endocardium;

FIG. 10 depicts the removal of the stylet after the fixation of the cardiac lead electrode in the endocardium;

FIG. 11 is a schematic diagram of a guide catheter for the implantation of the cardiac lead of FIG. 4;

FIG. 12A-FIG. 12B depict the guide catheter of FIG. 11 with a bent distal end;

FIG. 13-FIG. 14 depict a flow diagram for the cardiac lead implantation process according to this invention; and

FIG. 15 is schematic diagram of a pacemaker device.

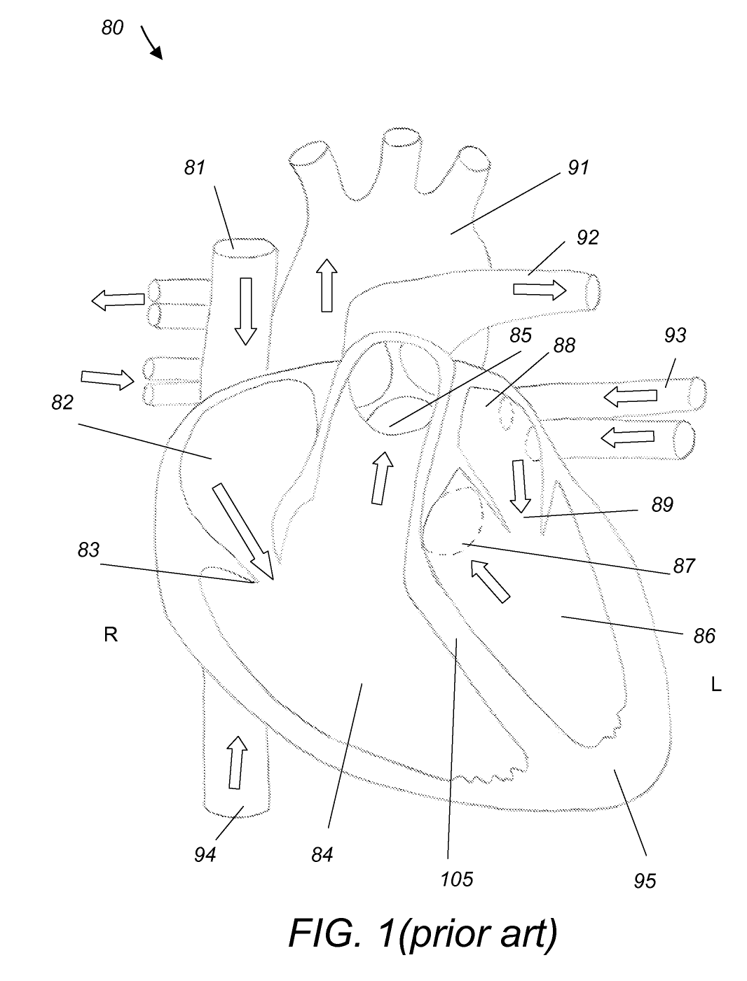

### DETAILED DESCRIPTION OF THE INVENTION

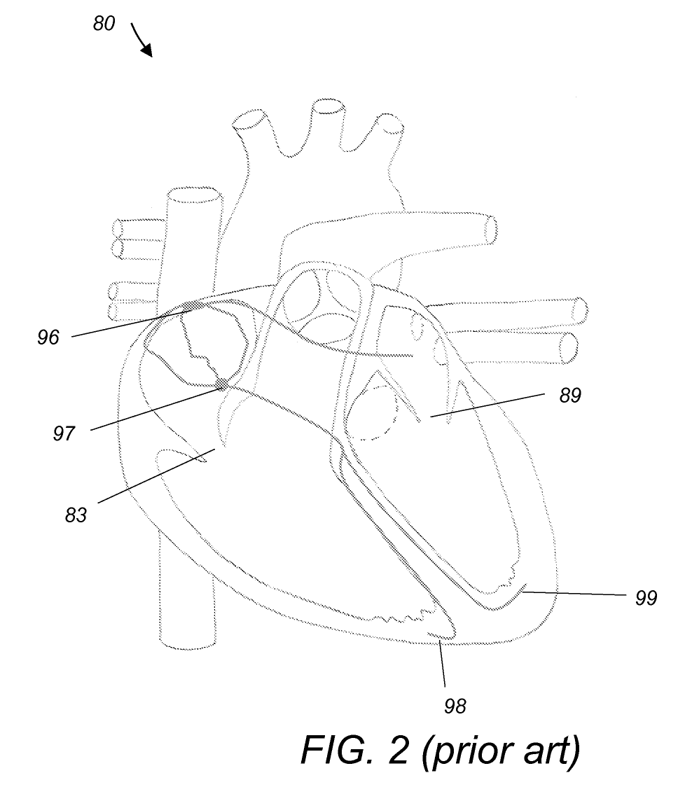

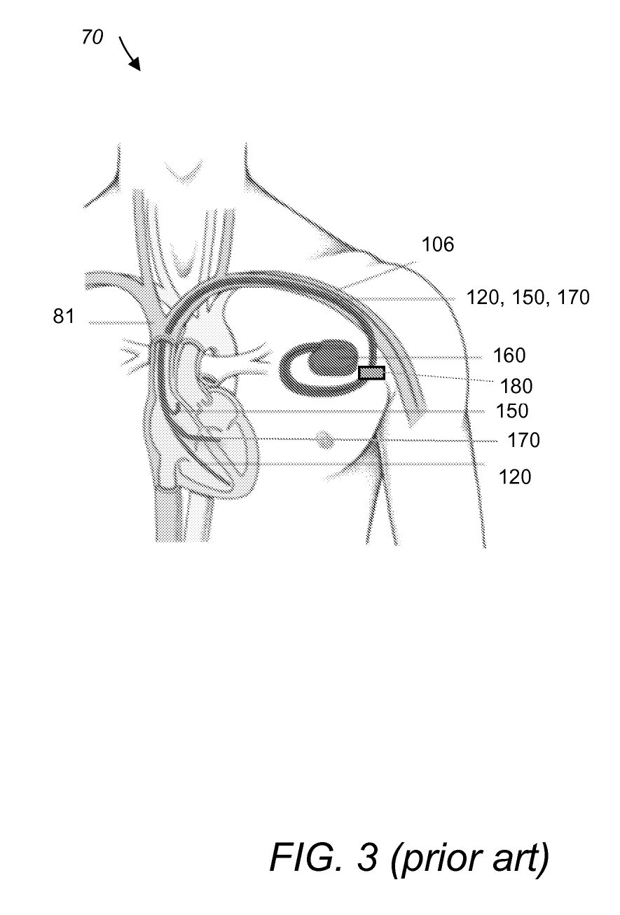

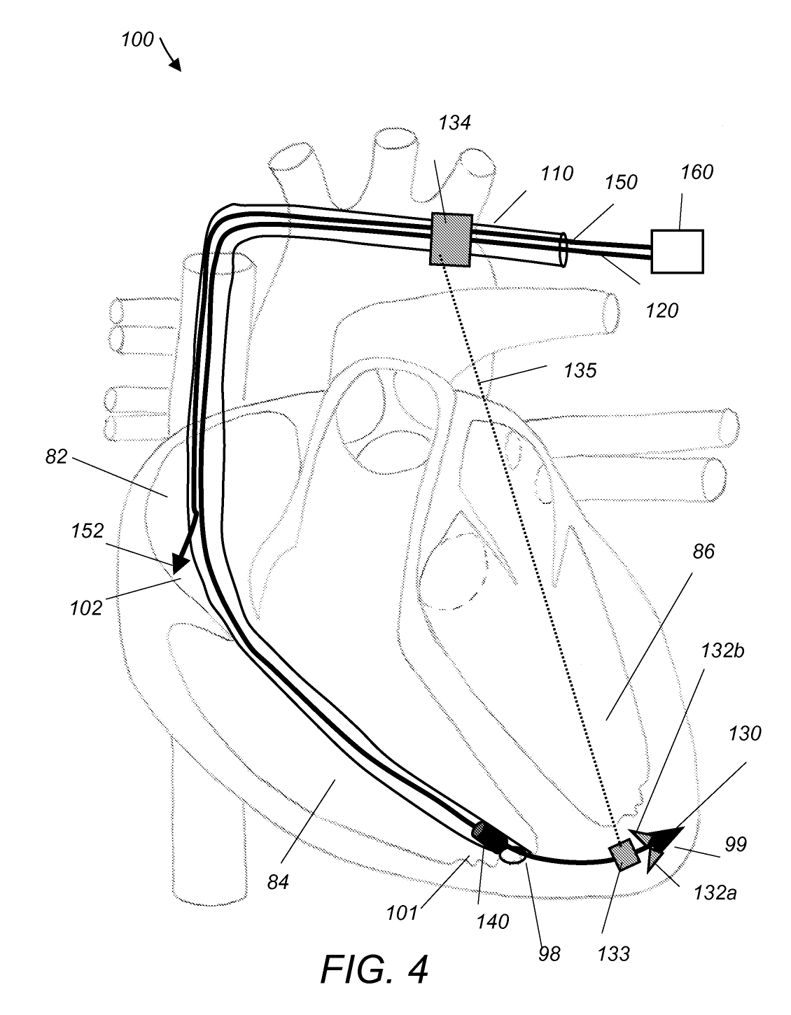

Referring to FIG. 4, pacemaker system 100 includes a pacemaker device 160, cardiac leads 120 and 150, guide catheter 110, an ultrasound transmitter 133 and an ultrasound receiver 134. Cardiac lead 150 is implanted in the right atrium 82 and comprises an electrode 152 at its distal end that is actively fixed into location 102 of the right atrium 82. Cardiac lead 120 is implanted in the right ventricle (RV) 84 and comprises a first electrode 140 (RV electrode) and a second electrode 130 (left ventricle (LV) electrode) at its distal end. First electrode 140 is positioned at a location 101 close to the apex 98 of the right ventricle 84. Second electrode 130 perforates the apex 98 of the right ventricle 84 and is actively fixed into the apex 99 of the left ventricle 86. This three electrode configuration 152, 140, 130, and their corresponding implantation locations 102, 101 and 99 are selected so that the artificial pacemaker system 100 simulates the efficacy and delivery locations of the heart's natural pacemaker, as shown and described in FIG. 2.

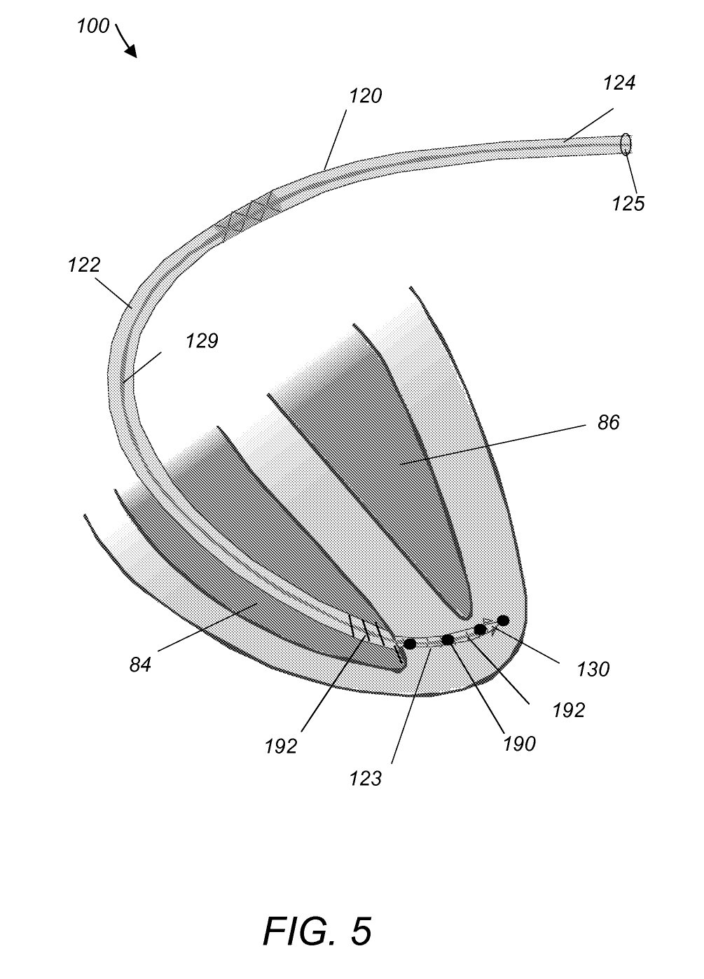

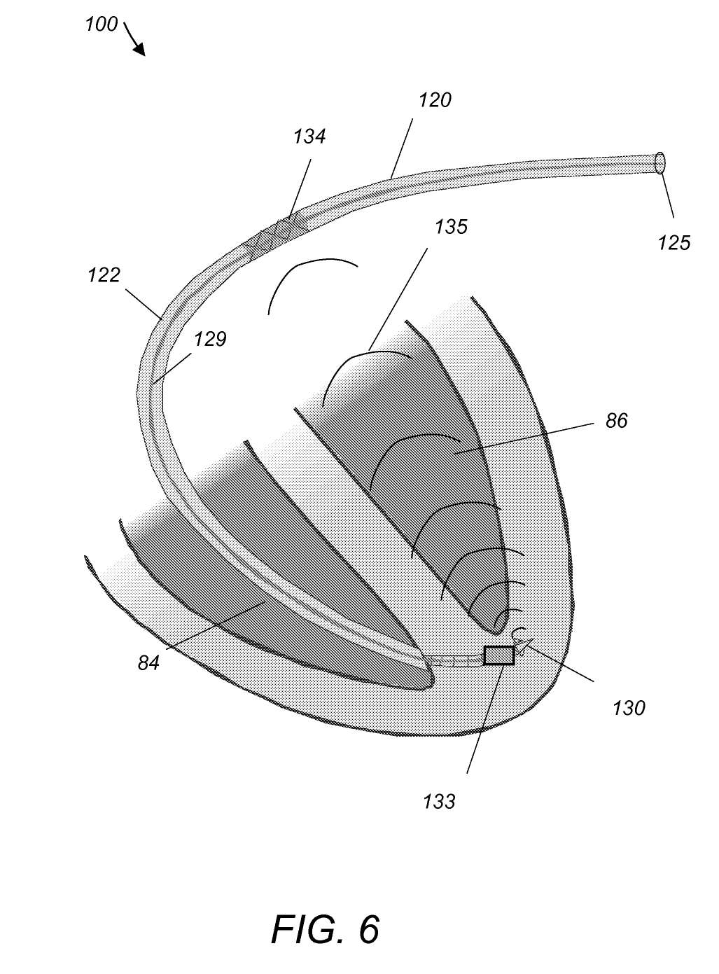

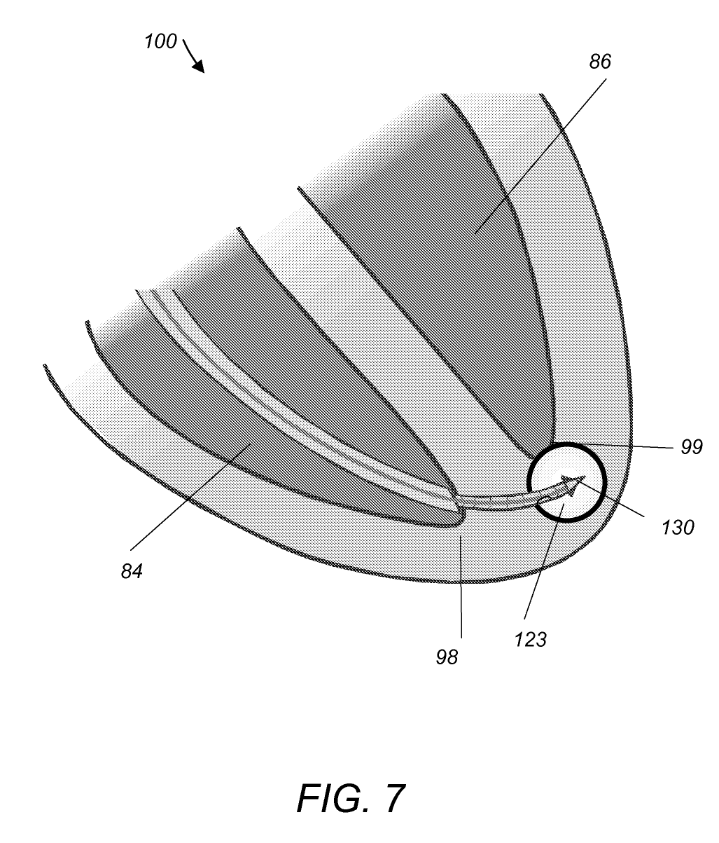

Referring to FIG. 5, cardiac lead 120 includes a flexible hollow tube 122 having a proximal end 124 and a distal end 123. Hollow tube 122 defines a lead lumen 125 that extends between the proximal and distal ends. The distal end 123 includes LV electrode 130 that is electrically connected to the pacemaker device 160 via a conductive element 129 extending through the lead lumen 125. In one example, tube 122 is made of conductive and/or heat shrinkable polymer and has a length of 50 cm and a diameter of 2 mm. In other examples the lead length may be in the range between 45 cm to 75 cm and its diameter may be in the range between 1 mm to 3 mm. The distal end 123 has a smaller diameter than the rest of the tube 122. After placement of the cardiac lead in the target area, tube 122 shrinks and its diameter decreases. In one example the diameter of the distal end 123 is 1 mm. Distal end 123 of tube 122 also includes apertures 190 which are used for delivering anti-inflammatory drugs or other therapeutic or diagnostic drugs to cardiac tissue, as will be described below.

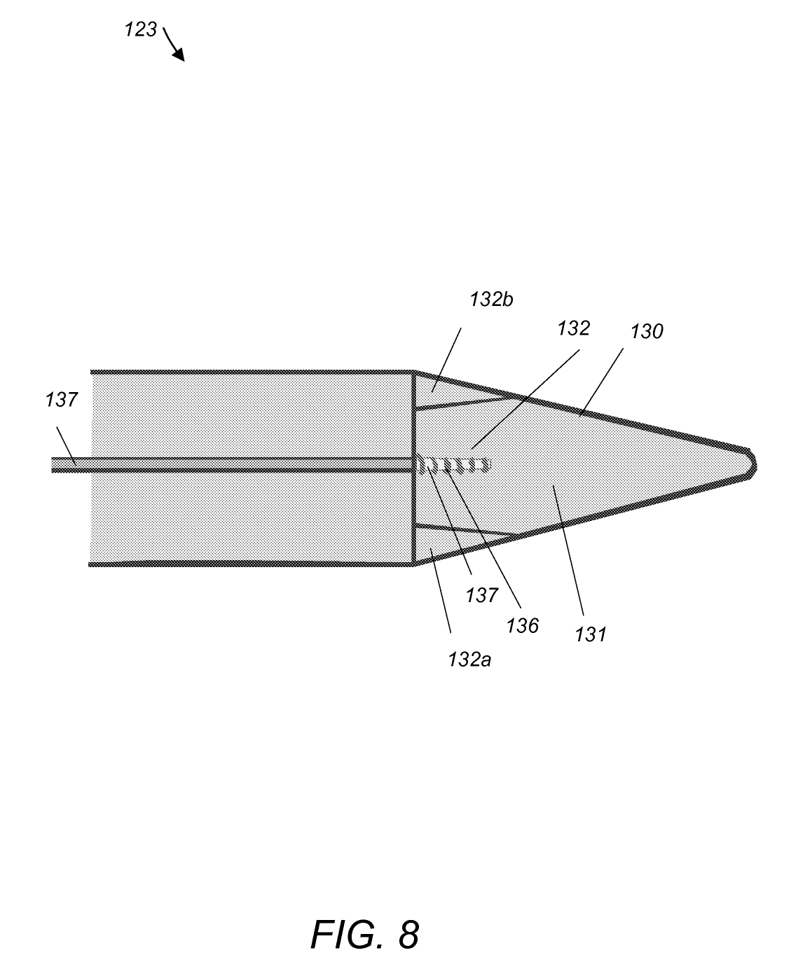

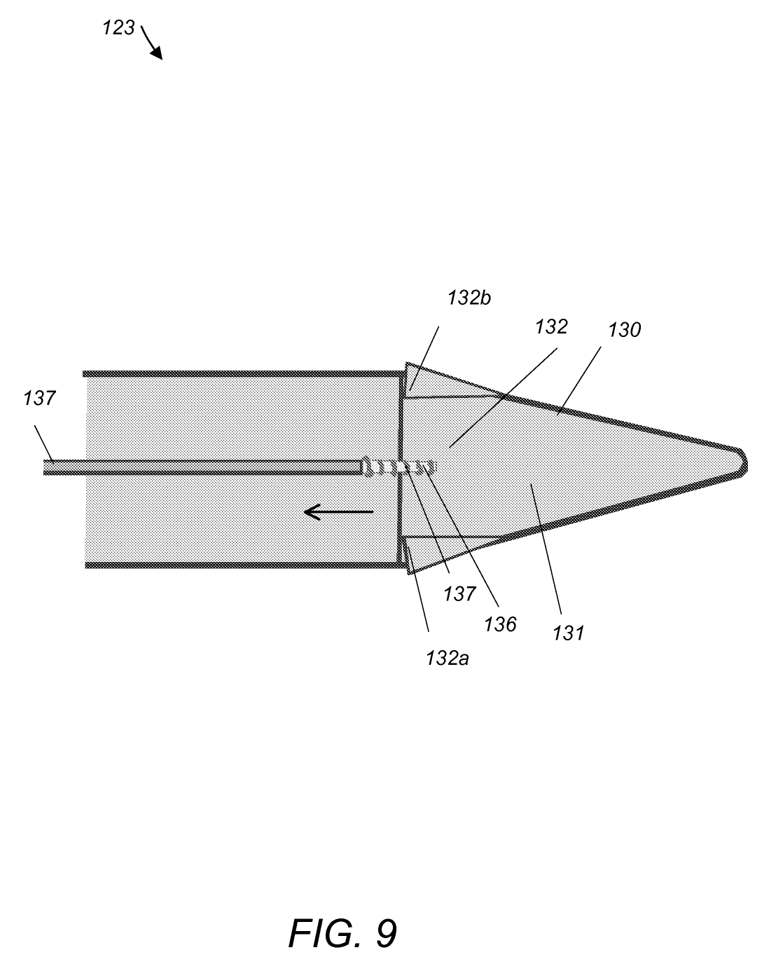

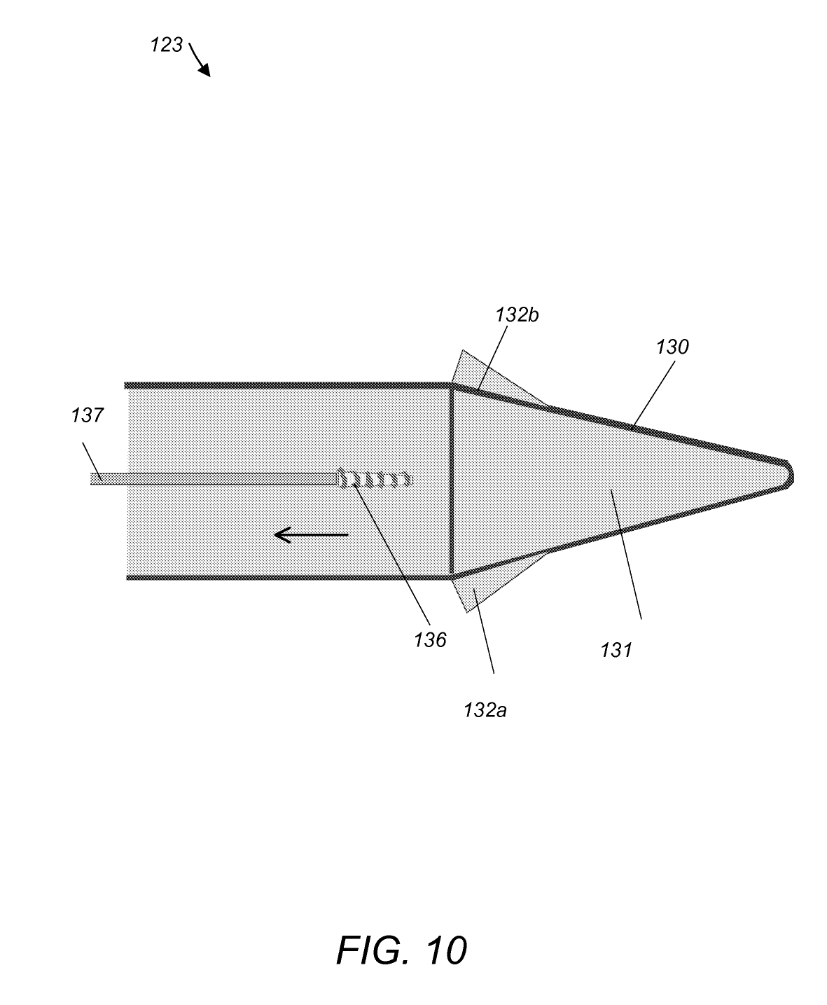

Referring to FIG. 8, distal end 123 of cardiac lead 120 includes a cone-shaped metal electrode 130. Electrode 130 is used for pacing and/or defibrillation of the left ventricle 86. Cone-shaped electrode 130 includes a cavity 131 that contains an active fixation mechanism 132. Active fixation mechanism 132 includes foldable wings 132a, 132b that are unfolded by activating the screw mechanism 136, as shown in FIG. 9 and FIG. 10. A stylet 137 is inserted through the lead lumen 125 and is attached onto the screw mechanism 136. Subsequently, the stylet 137 is used to activate the screw mechanism 136, thereby causing the wings 132a, 132b to unfold and fixate the electrode head 130 to a chosen location of the heart. In one example, stylet 137 is attached to the screw mechanism 136 via a clockwise rotation and the screw mechanism 136 is then activated via a counter-clockwise rotation. In other examples, the stylet 137 is pushed and snapped into the screw mechanism 136 and then pulled out to activate the screw mechanism 136. In other embodiments, the cardiac lead 120 includes a spiral type electrode.

Referring back to FIG. 4, the distal end 123 of the cardiac lead 120 includes an ultrasound transmitter 133 and the upper portion of the lead includes an ultrasound receiver 134. The ultrasound transmitter 133 sends ultrasound waves 135 that pass through the left ventricle 86 and the left atrium 88 and then are received by the ultrasound receiver 134. The ultrasound signal 135 is modulated by the heart rhythm and the signal received by the ultrasound receiver 134 contains information about the various heart properties including among others, the rhythm of both ventricles, heart rate, left ventricular ejection fraction (LVEF), left ventricular ejection time (LVET), left ventricular pre-ejection time (LPEP), global interval, cardiac output (CO) interval, EA interval (time interval between the diastolic blood flow velocity filling wave (E-wave) and the atrial blood flow velocity filling wave (A-wave)), Q-A2 interval, aortic velocity time integrals left ventricular pressure derivative (LVdp/dt), cardiac index (CI), cardiac output and fractional shortening. This signal is analyzed and transmitted to the pacemaker device 160 where it is used as feedback and control signal for controlling the pacing and defibrillation activities of the pacemaker device. Furthermore, the information from the feedback signal is also transmitted wirelessly to a location remote from the patient's locations for monitoring purposes. In one example, the remote location is a hospital, a doctor's office or any other remote monitoring facility. In other embodiments, the distal end 123 of the cardiac lead 120 includes a radio frequency (RF) transmitter (not shown) that sends RF signals to a receiver (not shown). The RF signal may also be used for controlling the pacemaker functions.

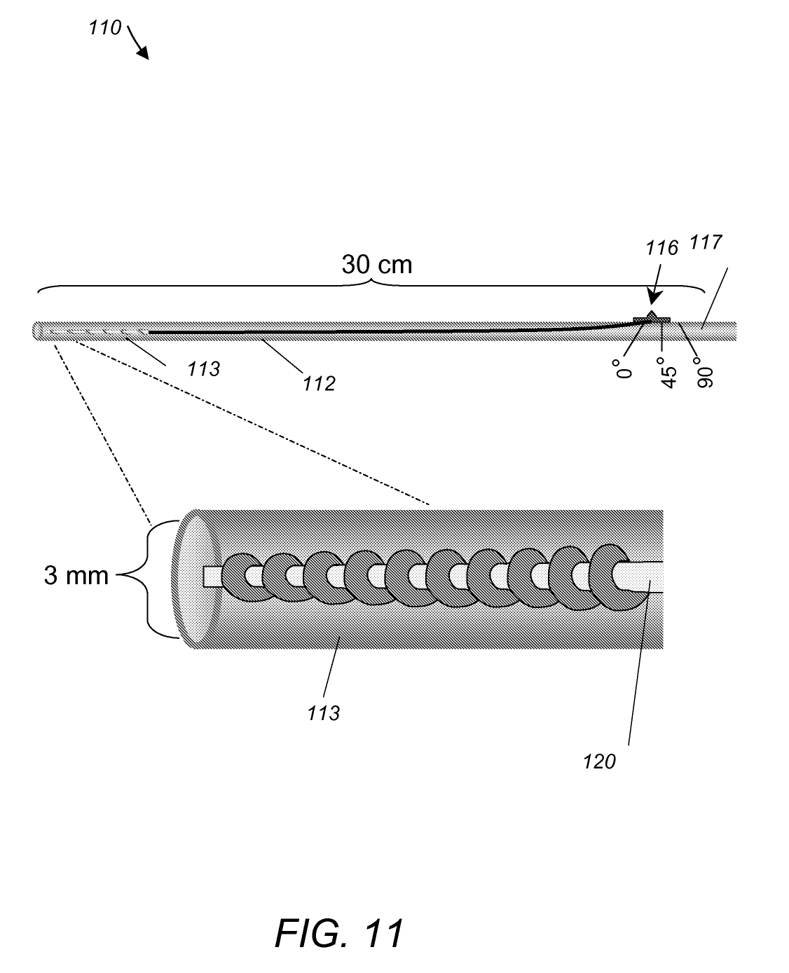

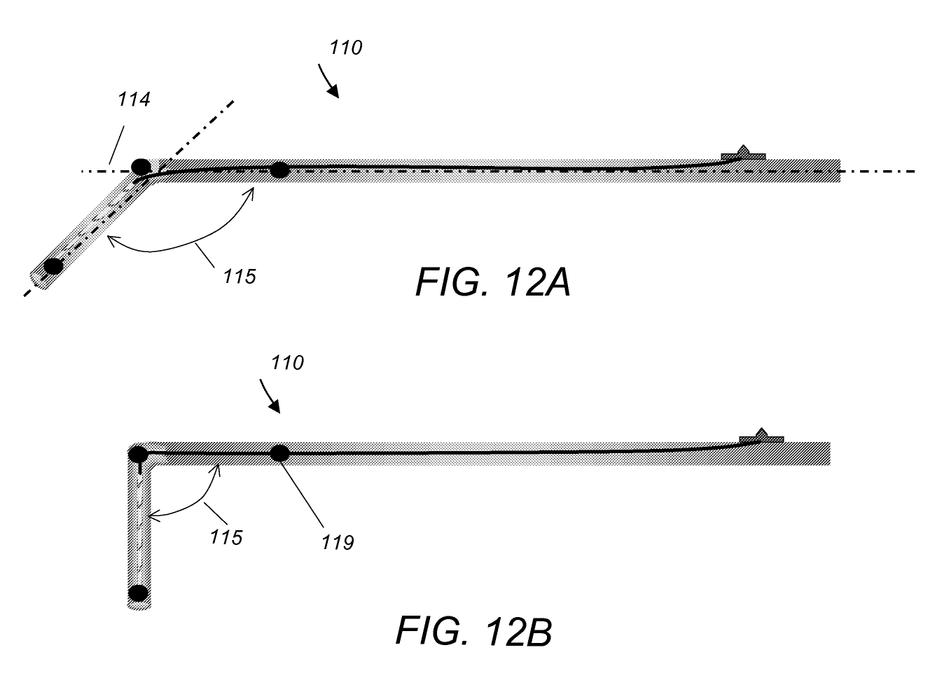

Cardiac leads 120 and 150 are implanted into the heart 80 with a guide catheter 110. Referring to FIG. 11, guide catheter 110 comprises a flexible hollow tube 112 that is inserted into the heart via the subclavian vein 106. Tube 112 includes a distal end 113 that is bendable so that it forms an angle 115 with the main axis 114 of the tube, as shown in FIG. 12A and FIG. 12B. Angle 115 is set via control 116 located at the proximal end 117 of tube 112. In one example, guide catheter 110 has a length of 30 cm, a diameter of 3 mm and is made of flexible material such as plastic or metal. Guide catheter 110 also includes radiographic position markers 119 for 3-D visualization and positioning. Guide catheter 110, cardiac leads 120, 150 and stylet 137 may be pre-assembled and implanted “en bloc”. In other embodiments, guide catheter 110 is inserted first and cardiac leads 120 and/or 150 are inserted afterward.

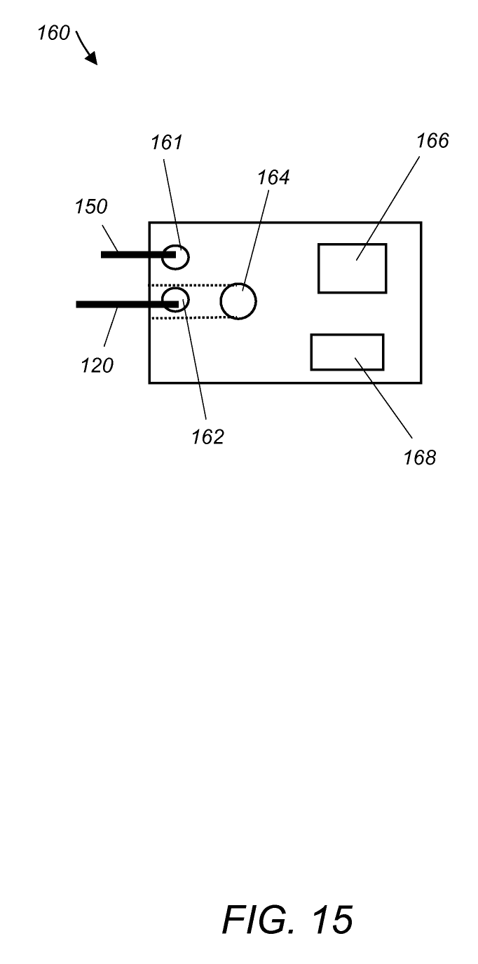

Referring to FIG. 15, pacemaker 160 includes two cardiac lead ports 161, 162, for connecting cardiac leads 150, 120, respectively, a drug injection port 164, a processor 166 and a wireless transmitter 168. Drug injection port 164 communicates with tube 122 of lead 120 and is used to inject drugs, stem cells dies, genes or any other medication substance to the heart muscle of the RV and/or LV via apertures 190. Processor 166 processes and controls the electrical signals sent to the electrodes and the feedback signals received from the ultrasound receiver 134. Wireless transmitter 168 transmits the information from the feedback signal wirelessly to a location remote from the patient's locations for monitoring purposes.

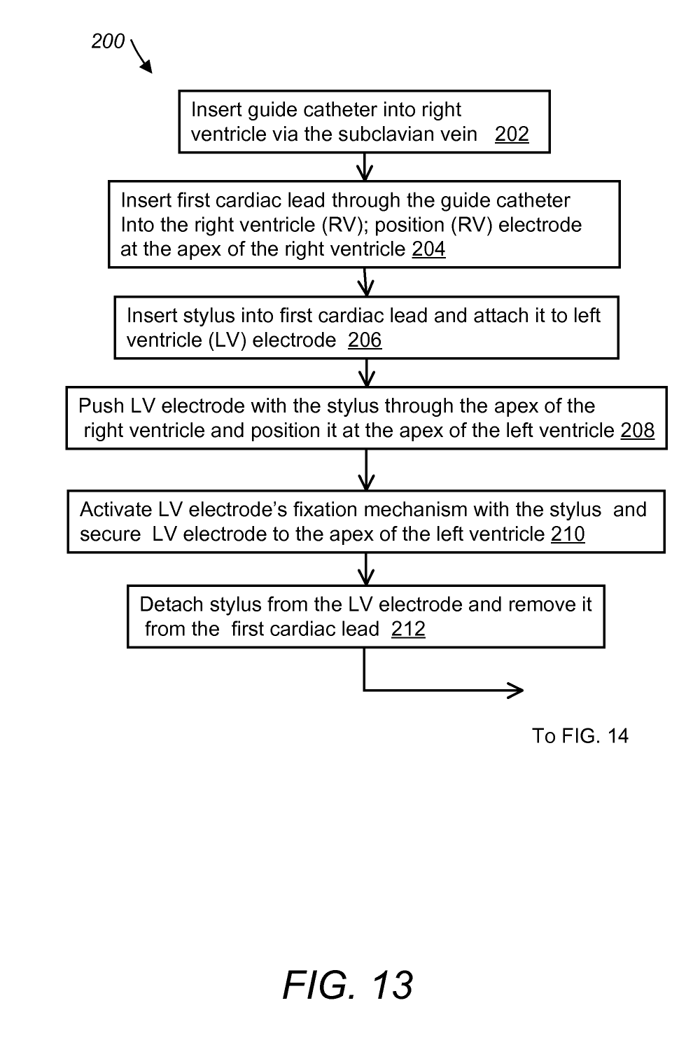

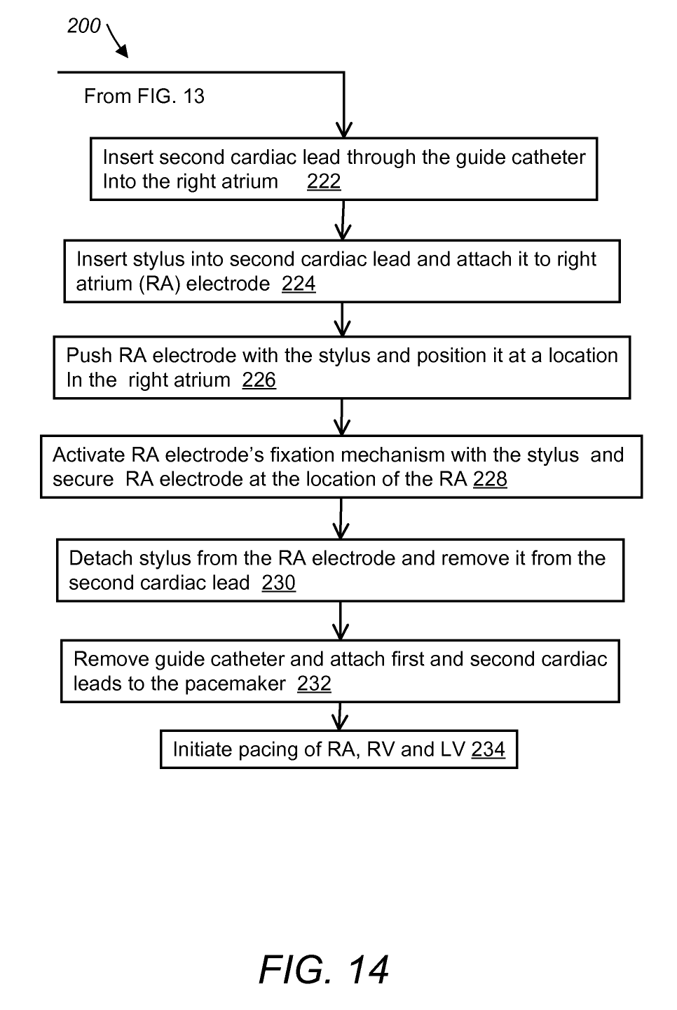

Referring to FIG. 13 the process 200 of implanting the cardiac leads 120, 50 into the heart includes the following steps. First a guide catheter 110 is inserted into the right ventricle (RV) 84 via the subclavian vein (202). The guide catheter 110 is steered and positioned within the RV in contact with the endocardium so that its distal tip is near the apex 98 of the RV 84. Next, a first cardiac lead 120 is inserted through the guide catheter 110 into the RV 84 (204) and is positioned so that the RV electrode 140 is near the apex 140 of the RV 84. As was mentioned above, in other embodiments, guide catheter 110 and cardiac leads 120, 150 may be pre-assembled and inserted into the RV 84 en bloc. Next, a stylet 137 is inserted into the first cardiac lead 120 and is attached to the left ventricle (LV) electrode 130 at the distal end of the first cardiac lead (206). Next, the LV electrode 130 is pushed with the stylet 137 through the apex 98 of the RV 84 and is positioned at the apex 99 of the LV 86 (208). Next, the LV electrode's fixation mechanism is activated with the stylet 137 and the LV electrode 130 is secured to the apex 99 of the LV 86(210). As was described above, the stylet 137 is attached to the LV electrode 130 via clockwise rotation of the stylet and the electrode's fixation mechanism is activated via counter clockwise rotation of the stylet. The fixation mechanism involves unfolding two or more foldable wings 132a, 132b. Continuing the counter clockwise rotation of the stylet 137 removes it from the electrode 130 (212). At the end of this procedure, cardiac lead 120 is positioned within the RV so that RV electrode 140 is near the apex 98 of the RV 84 and LV electrode 130 is fixated at the apex 99 of the LV 86. Next, the proximal end of cardiac lead 120 is connected to the pacemaker device 160 and biventricular pacing is initiated.

If separate pacing of the right atrium (RA) is required, a second cardiac lead 150 is inserted through the catheter 110 into the RA 82 (222). The stylet 137 is then inserted into the second cardiac lead 150 and is attached to the RA electrode 152 at the distal end of the second cardiac lead 150 (224). The RA electrode 152 is pushed and positioned with the stylet at a location 102 of the RA 82 (226) and the RA electrode's fixation mechanism is activated (228). Once the RA electrode 152 is secured at the chosen location 102 of the RA 82, the stylet 137 is detached from the RA electrode 152 and is removed from the second cardiac lead 150 (230). Next, the guide catheter is removed and the proximal ends of the first and second cardiac leads are attached to the pacemaker device (232). Finally, pacing of the RA 82, RV 84 and LV 86 is initiated (234).

Unlike a conventional cardiac resynchronization therapy (CRT) system that requires three different cardiac leads (one for the left ventricle, one for right ventricle, and one for the right atrium), the current design combines the right and left ventricular leads into one lead. This lead design enables fast implantation and shortens the overall time of the surgical procedure. As was mentioned above, in the prior art CRT systems, the left ventricle stimulation lead is usually introduced either through cannulation of the coronary sinus, or from the right ventricle through perforation of the septum. Cannulation of the coronary sinus may cause blood flow, obstructions and problems with the fixation of the lead. The implanted cardiac leads may become dislodged either during the implantation procedure or by normal physiological activity of the patient. A clip-on tool and/or other fixation devices, such as screws or anchors are used for the fixation of the leads and the prevention of lead dislodgement. The installation of these additional components increases the duration and complexity of the surgery. The present lead design eliminates these problems. Furthermore, the active fixation mechanism of the present lead design secures a reliable placement of the electrodes in the target tissue area and eliminates the lead dislodgement problems of the prior art leads. The implantation of two leads instead of three leads reduces the risk of infection. The specific lead design and fixation mechanism eliminates the need for a clip-on fixation tool. Simultaneous pacing of the RV and/or the LV is accomplished. The cost of the pacemaker system is reduced because only one lead is used for both ventricles instead of two. The cardiac lead does not cause abnormal diaphragm stimulation. The chosen locations of the two separate electrodes are considered optimal positions for bi-ventricular stimulation.

Other embodiments may include one or more of the following. The electrode fixation mechanism may be an expanding wedge or other diametrically expanding structure. The guide catheter may also include ultrasound transmitting devices for positioning purposes. The drug injection port may be marked with radio-opaque material which is visible under fluoroscopy. The outer surface of the drug injection port may be crater-shaped, which permits easy and quick attachment of the injection needle. The pacemaker battery may be charged remotely from outside the body, thus eliminating the need for battery replacement surgery. In other embodiments the battery is recharged via the heart beating or other body movement. The outer surfaces of cardiac leads 120, 150 or the outer surfaces of electrodes 130, 140, 152 may include wire coil electrodes 192 (shown in FIG. 5) for applying an electric shock to the cardiac muscle of both ventricles.

Several embodiments of the present invention have been described. Nevertheless, it will be understood that various modifications may be made without departing from the spirit and scope of the invention. Accordingly, other embodiments are within the scope of the following claims.

What is claimed is:

1. A cardiac pacemaker system comprising:
a pacemaker device comprising a pulse generator for producing cardiac stimulating pulses;
a first cardiac lead connected to said pulse generator and comprising first and second electrodes and being shaped and dimensioned to be implanted in the right cardiac ventricle;
wherein said first electrode comprises first fixation means for actively fixing said first electrode to the apex of the right cardiac ventricle and is used for pacing, sensing and/or defibrillating of the right cardiac ventricle;
wherein said second electrode comprises means for penetrating the apex of the right cardiac ventricle, means for entering into the left cardiac ventricle and second fixation means for actively fixing said second electrode to the apex of the left cardiac ventricle and is used for pacing, sensing and/or defibrillating of the left cardiac ventricle;
a second cardiac lead connected to said pulse generator and comprising a third electrode and being shaped and dimensioned to be implanted in the right cardiac atrium and is used for pacing, sensing and/or defibrillating of the right cardiac atrium;
wherein said stimulating pulses are transmitted to said right and left cardiac ventricles via said first cardiac lead and to said right cardiac atrium via said second cardiac lead and stimulate said apex of the right cardiac ventricle, said apex of the left cardiac ventricle and said first location of the right cardiac atrium via said first, second and third electrodes, respectively.

2. The cardiac pacemaker system of claim 1 wherein said first cardiac lead comprises a flexible hollow tube comprising a proximal end and a distal end and wherein said hollow tube defines a lumen extending between said proximal and distal ends and is dimensioned to house a conductive lead connecting said pulse generator to said first and second electrodes.

3. The method of claim 2 wherein said conductive lead comprises a conductive heat shrinkable polymer.

4. The cardiac pacemaker system of claim 2 wherein said second electrode comprises a cone-shaped body having a sharp tip end for penetrating the apex of the right cardiac ventricle and a cavity containing said second fixation means.

5. The cardiac pacemaker system of claim 4 wherein said second fixation means comprise first and second foldable wings, a screw-driven mechanism for folding and unfolding said first and second foldable wings and a stylet used to activate said screw-driven mechanism and to push said second electrode into the left cardiac ventricle and wherein said stylet is inserted through said lumen and is attached to said screw-driven mechanism.

6. The cardiac pacemaker system of claim 4 wherein said second fixation means comprise an expandable wedge with first and second diametrically expandable components, a screw-driven mechanism for expanding or contracting said first and second expandable components and a stylet used to activate said screw-driven mechanism and to push said second electrode into the left cardiac ventricle and wherein said stylet is inserted through said lumen and is attached to said screw-driven mechanism.

7. The cardiac pacemaker system of claim 5 wherein said stylet is attached to said screw-driven mechanism via a clockwise rotation and activates said screw-driven mechanism via a counter-clockwise rotation.

8. The cardiac pacemaker system of claim 5 wherein said stylet is pushed forward to be attached to said screw-driven mechanism and is pulled back to activate said screw-driven mechanism.

9. The cardiac pacemaker system of claim 2 wherein said first cardiac lead further comprises an ultrasound transmitter at its distal end and an ultrasound receiver at its proximal end and wherein said ultrasound transmitter is located and oriented so that it transmits ultrasound waves that pass through the cardiac left ventricle and left atrium and are modulated by the cardiac rhythm prior to being received by the ultrasound receiver.

10. The cardiac pacemaker system of claim 9 wherein the modulated ultrasound waves comprise information about at least one of rhythm of left and right cardiac ventricles, heart rate, left ventricular ejection fraction, left ventricular ejection time, left ventricular pre-ejection time, global interval CO interval, EA interval, Q-A2 interval, aortic velocity time integrals LVdp/dt, CI, cardiac output and fractional shortening and wherein said pacemaker device comprises a processor for analyzing said information and providing feedback control to the pulse generator.

11. The cardiac pacemaker system of claim 10 wherein said pacemaker device further comprises a wireless transmitter for transmitting said information wirelessly to a remote location for monitoring purposes.

12. The cardiac pacemaker system of claim 5 further comprising a guide catheter, wherein said guide catheter comprises a flexible hollow tubular body dimensioned to house said first and second cardiac leads and said stylet and to be implanted into a mammalian heart via the subclavian vein and wherein said tubular body comprises a distal end that is bendable and forms an angle with the tube main axis and wherein said angle is controlled via a control located at the proximal end of the tubular body.

13. The cardiac pacemaker system of claim 12 wherein said guide catheter further comprises radiographic position markers for 3-D visualization and positioning.

14. The cardiac pacemaker system of claim 12 wherein said guide catheter further comprises diagnostic devices for determining the condition of the surrounding cardiac tissue.

15. The cardiac pacemaker system of claim 12 wherein said pacemaker device further comprises a drug injection port that connects to the flexible hollow tube of the first cardiac lead and is used to inject drugs, stem cells dies, genes or other medication substance to the right cardiac ventricle and/or the left cardiac ventricle.

16. The cardiac pacemaker system of claim 15 wherein said first and second electrodes comprise apertures for delivering said drugs to the right cardiac ventricle and/or the left cardiac ventricle, respectively.

17. A method of stimulating a mammalian heart via a single pacing/sensing cardiac pacemaker system comprising:
providing a pacemaker system comprising a pacemaker device first and second cardiac leads and a guide catheter wherein said pacemaker device comprises a pulse generator for producing cardiac stimulating pulses, wherein said first cardiac lead is connected to said pulse generator and comprises first and second electrodes, wherein said second cardiac lead is connected to said pulse generator and comprises a third electrode, and wherein said guide catheter comprises a flexible hollow tubular body dimensioned to house said first and second cardiac leads and a stylet;
inserting said guide catheter into a right cardiac ventricle via the subclavian vein;
inserting said first cardiac lead through said guide catheter into the right cardiac ventricle and actively fixing said first electrode to the apex of the right cardiac ventricle with first fixation means;
inserting said stylet into the first cardiac lead and attaching said stylet to the second electrode;
pushing said second electrode with the stylet through the apex of the right cardiac ventricle and position it at the apex of the left cardiac ventricle;
activating the second electrode's active fixation mechanism with the stylet and fixing said second electrode to the apex of the left cardiac ventricle with second fixation means;
inserting said second cardiac lead through the guide catheter into the right cardiac atrium;
initiating stimulation of the right cardiac ventricle, left cardiac ventricle and right cardiac atrium via said first second and third electrodes, respectively, wherein said stimulation comprises at least one of pacing, sensing or defibrillation.

18. The method of claim 17 wherein said first cardiac lead comprises a flexible hollow tube comprising a proximal end and a distal end and wherein said hollow tube defines a lumen extending between said proximal and distal ends and being dimensioned to house a conductive lead connecting said pulse generator to said first and second electrodes.

19. The method of claim 18 wherein said second electrode comprises a cone-shaped body having a sharp tip end for penetrating the apex of the right cardiac ventricle and a cavity containing said second fixation means.

20. The method of claim 19 wherein said second fixation means comprise first and second foldable wings, a screw-driven mechanism for folding and unfolding said first and second foldable wings and wherein said stylet is inserted through said lumen and is attached to said screw-driven mechanism and is used to activate said screw-driven mechanism and to push said second electrode into the left cardiac ventricle.

21. The method of claim 18 wherein said first cardiac lead further comprises an ultrasound transmitter at its distal end and an ultrasound receiver at its proximal end and wherein said ultrasound transmitter is located and oriented so that it transmits ultrasound waves that pass through the cardiac left ventricle and left atrium and are modulated by the cardiac rhythm prior to being received by the ultrasound receiver.

22. The method of claim 21 wherein the modulated ultrasound waves comprise information about at least one of rhythm of left and right cardiac ventricles, heart rate, left ventricular ejection fraction, left ventricular ejection time, left ventricular pre-ejection time, global interval, CO interval, EA interval, Q-A2 interval, aortic velocity time integrals LVdp/dt, CI, cardiac output and fractional shortening and wherein said pacemaker device comprises a processor for analyzing said information and providing feedback control to the pulse generator.

23. The method of claim 22 wherein said pacemaker device further comprises a wireless transmitter for transmitting said information wirelessly to a remote location for monitoring purposes.

24. The method of claim 17 wherein said tubular body of said guide catheter comprises a distal end that is bendable and forms an angle with the tube main axis and wherein said angle is controlled via a control located at the proximal end of the tubular body.

25. The method of claim 24 wherein said guide catheter further comprises radiographic position markers for 3-D visualization and positioning.

26. The method of claim 24 wherein said pacemaker device further comprises a drug injection port that connects to the flexible hollow tube of the first cardiac lead and is used to inject drugs, stem cells dies, genes or other medication substance to the heart muscle of the right ventricle and/or the left ventricle.
